(19) United States
(12) Patent Application Publication
(10) Pub. No.: US 2022/0395298 A1
(43) Pub. Date: Dec. 15, 2022

(54) Modular Bone Anchor Assembly

(71) Applicant: Anza Innovations, Inc., Del Mar, CA (US)

(72) Inventor: Eugene Shoshtaev, Del Mar, CA (US)

(73) Assignee: Anza Innovations, Inc., Del Mar, CA (US)

(21) Appl. No.: 17/839,460

(22) Filed: Jun. 13, 2022

Related U.S. Application Data
(60) Provisional application No. 63/209,765, filed on Jun. 11, 2021.

(51) Int. Cl. A61B 17/70; A61B 17/86

(52) U.S. Cl. CPC A61B 17/7037; A61B 17/7032; A61B 17/8685

(57) ABSTRACT

The disclosure provides a spinal implant having spinal anchors comprising a bone anchor and a receiver configured to securely and polyaxially receive the bone anchor. Further, provided herein are methods for using the spinal implant.

### CROSS-REFERENCE TO RELATED APPLICATIONS

The present application is a non-provisional patent application claiming the benefit of priority under 35 U.S.C. § 119(e) to U.S. Provisional Patent Application Ser. No. 63/209,765, filed Jun. 11, 2021 and entitled “Modular Bone Anchor Assembly,” the complete disclosure of which is hereby incorporated by reference as if set forth fully herein.

### FIELD

The present disclosure relates generally to surgical fixation systems, and in particular to polyaxial anchor assemblies configured for use with orthopedic stabilization systems.

### BACKGROUND

Spinal deformities, injuries, and diseases involving the spine have been treated surgically using anchor assembly spinal implants comprising a plurality of anchors assemblies and a rod, whereby the substantially rigid rod connects the anchor assemblies to form a structure that supports, realigns, and/or repositions certain vertebrae for the purpose of stabilizing, immobilizing, and/or adjusting spinal alignment.

### SUMMARY

Disclosed herein is a bone anchor assembly according to some embodiments. In some embodiments, the bone anchor assembly includes an anchor member, a receiver, compression element, and a locking element. In some embodiments, the bone anchor assembly may be configured to accept an elongate stabilizer (i.e., a rod) into the receiver, which forms a multi-axial locked relationship with the anchor member (e.g., a screw, hook, nail, staple, etc.). The anchor member may be advantageously capable of being either top loaded or bottom loaded into a receiver and the receiver may be rotated into a position for holding the elongate stabilizer with the compression member that captures a spherical shaped head of the anchor member at one of many possible angles. The elongate stabilizer may be loaded into the receiver, unto the compression member, and held in that position by a locking element that threads into or onto the receiver and secures the construct in the desired position. In some embodiments, the bone anchor assembly of the present disclosure may be substantially similar to the anchor assemblies described in commonly owned U.S. patent application Ser. No. 16/049,674, filed on Jul. 30, 2018 and entitled “Spinal Stabilization System Including Bottom Loading Wide Angulation Polyaxial Rod Anchor Assemblies” (and issued as U.S. Pat. No. 11,076,888 on Aug. 3, 2021) the complete disclosure of which is hereby incorporated by reference into this disclosure as if set forth fully herein.

In some embodiments, the anchor member may include a proximally oriented head portion, a distally oriented elongated shank extending axially from said head along a longitudinal axis, and a neck portion positioned between the head and the shank. In some embodiments, the head may comprise a spherically shaped head having a curved outer surface and a proximal head recess that truncates the spherical shape of the head. In some embodiments, the head recess comprises a channel. In some embodiments, the channel is generally perpendicular to the longitudinal axis of the anchor. In some embodiments, the head recess may have a concave curvature resulting in an outer perimeter having a pair of opposing raised portions on either side of the channel. The neck may have a diameter of less than a diameter of the head. In some embodiments, the shank includes a bone engagement feature. In some embodiments, the bone engagement feature comprises a helical thread extending at least substantially the length of the shank, the helical thread being configured to engage bone. In some embodiments, the distal end of the shank may comprise a narrow tip to facilitate entry into bone. In some embodiments, at least a portion of the shank may comprise a feature to affix to a separate component having a bone engagement feature.

In some embodiments, the head may further comprise one or more distal recesses that function to enable a keyed insertion of the anchor member into the receiver.

In some embodiments, the bone anchor may further comprise one or more reliefs that increase the span at which the bone anchor can rotate with respect to the receiver.

In some embodiments, the anchor member may be cannulated such that the shank comprises an inner lumen extending between the head and the distal tip of the shank.

In some embodiments, the receiver may have a generally squared-off U-shaped profile with a partially cylindrical inner profile and a substantially faceted cylindrical outer profile. Alternatively, the outer profile could also be of another configuration, for example, curved, faceted, or rectilinear.

In some embodiments, the receiver includes a base and pair of spaced and generally parallel arms that form an open generally U-shaped channel therebetween that may be open at proximal ends of the arms.

In some embodiments, the receiver further includes a chamber or cavity located within the base communicating with and located beneath or distal to the U-shaped channel. In some embodiments, the chamber or cavity includes a substantially spherical seating surface portion configured to seat or receive the head of the anchor member.

In some embodiments, the base of the receiver further comprises a distal surface and a distal or terminal opening in communication with the seating surface portion and the cavity. The distal opening is substantially coaxially aligned with respect to a rotational axis of the receiver (e.g., an axis extending longitudinally through the U-shaped channel and cavity in a proximal to distal direction). In some embodiments the distal opening has a generally circular shape that may be partially occluded by a keyed feature or shoulder extending into the circular shape of the distal opening. By way of example, the shoulder may be configured to aid in retention of the anchor member. In some embodiments, the bottom edge of the terminal opening may include chamfers that allow increased angulation of the shank in preferred directions when assembled into receiver.

In some embodiments, base of the receiver further includes a cavity slot that communicates with the contoured volume of the cavity, as well as the distal opening and a receiver side exterior. In some embodiments, the cavity slot is located opposite the shoulder. In some embodiments, the cavity slot is shaped to allow the neck of the anchor member to reside therein. In some embodiments, the receiver further comprises one or more interference elements in the form of protrusions positioned on the bottom edge of the terminal opening at the interface with the cavity slot.

In some embodiments, the cavity slot allows the anchor member to assume an entry position during bottom-loading assembly (and/or disassembly) of the anchor assembly where the longitudinal axis of the anchor member is generally perpendicular to the receiver rotational axis, the head recess of the anchor member is aligned and engaged with the shoulder of the receiver, and one of the distal recesses of the head is engaged with one of the protrusions of the receiver. With the engagement of the protrusion within the distal recess and one of the sidewalls acting as a buttress, the anchor member may be rotated about its longitudinal axis, enabling the neck to rotate or “roll” into the cavity slot and the head of the anchor member to pass both the shoulder and the protrusions into (and/or out of) the contoured volume of the cavity. Once the head is seated within the contoured volume of the cavity, the anchor member may be pivoted in many different directions or angles without dissembling from the receiver.

In some embodiments, the compression element includes a body having a substantially circular cross section and a pair of upstanding arms extending in a proximal direction from the body. In some embodiments, the body and arms form a generally U-shaped, open channel having a substantially U-shaped bottom rod seating surface. In some embodiments, each upstanding arm may further include a relief cut designed to thin the arms and allow them to flex slightly during assembly of compression element into the receiver. In some embodiments, the compression element further includes a curved or spherical inner surface sized and shaped to frictionally engage and mate with the head of the anchor member.

In some embodiments, the compression element may be top-loaded into the receiver by means of the upstanding arms deflecting slightly inward during assembly as the compression element is advanced into the receiver until protrusions on the compression element reach retention features (i.e. recesses) of the receiver, thereby allowing the upstanding arms to spring outward and engage the retention features. At this stage, the compression element is detained inside the receiver and prevented from coming back out through the top of the receiver, while also being allowed some ability to translate distally under load in order to press upon the head of the anchor member, with or without deflection of the arms.

In some embodiments, when fully assembled, with the anchor element coupled with the receiver, a rod or connecting member is inserted through the U-shaped channel of the receiver until it is pressing on the rod seating surface of the compression element. This distal force exerted on and by the rod causes the spherical inner surface of the compression element to frictionally engage the curved outer surface of the head of the anchor element which in turn presses upon the seating surface portion of the receiver. In some embodiments, that the compression element may take on a variety of different configurations and means of retention inside the receiver.

In some embodiments, the compression element may include additional features configured to increase the frictional interface between the head of the anchor member and the receiver.

In some embodiments, the locking element includes a compression bottom surface that presses against the connecting member that in turn presses upon the compression element that in turn presses the head of the anchor member into fixed frictional contact with the receiver, so as to lock or fix the connecting member relative to the vertebra. In some embodiments, the receiver and the anchor member cooperate in such a manner that the receiver and the anchor member can be secured at any of a plurality of angles, articulations, or rotational alignments relative to one another and within a selected range of angles both from side to side and from front to rear, to enable flexible or articulated engagement of the receiver with respect to the anchor member until both are locked or fixed relative to each other near the end of an implantation procedure.

In some embodiments, the anchor member may be advantageously capable of being either top loaded or bottom loaded into a receiver and the receiver may be rotated into a position for holding the elongate stabilizer with the compression member that captures a spherical shaped head of the anchor member at one of many possible angles.

In some embodiments, the compression element includes a body having a substantially circular cross section, a pair of upstanding arms extending in a proximal direction from the body, and a distal engagement portion extending distally from the body. In some embodiments, the body and arms form a generally U-shaped, open channel having a substantially U-shaped bottom rod seating surface. In some embodiments, each upstanding arm may further include a relief cut designed to thin the arms and allow them to flex slightly during assembly of compression element into the receiver.

In some embodiments, the distal engagement portion is configured to receive a substantial portion of the head of the anchor member therein. In some embodiments, the distal engagement portion comprises a plurality of curved, flexible flanges arranged in an annular ring configuration and defining a cavity therein sized and configured for receiving the head of the anchor member therein. In some embodiments, the distal ends of the flexible flanges together form an annular opening into the cavity. In some embodiments, the flanges are each curved such that each flange has an inner surface having a concave curvature and an outer surface having a convex curvature, and further such that the maximum diameter of the cavity is greater than the diameter of the opening. By way of example, the diameter of the opening in a first, nonexpanded state is smaller than the maximum diameter of the head such that, absent expansion of the opening, the head is prevented from entering or exiting the cavity.

In some embodiments, the annular ring of flexible flanges is configured to flex or expand radially outward to widen the opening and allow passage of the head of the anchor member through the expanded opening in response to a proximal or upward force exerted by the head on the compression element, for example when the anchor member is being coupled to the receiver through a bottom-loading coupling process.

In some embodiments, once the head of the anchor member has been inserted into the receiver (by virtue of coupling with the compression element), a distal oriented or downward force applied to the anchor member causes the compression element to translate distally within the receiver (and correspondingly causes the protrusions to translate distally within the retention features). As a result of this distal translation, the flexible flanges are blocked from flexing or expanding radially outward by virtue of the inner cylindrical surface of the receiver cavity. Thus, the head of the anchor member is captured within the cavity of the compression element and the anchor member is securely coupled with the receiver.

### BRIEF DESCRIPTION OF THE DRAWINGS

Many advantages of the present disclosure will be apparent to those skilled in the art with a reading of this specification in conjunction with the attached drawings, wherein like reference numerals are applied to like elements and wherein:

FIG. 1 is a perspective view of an example of a bone anchor assembly according to some embodiments;

FIG. 2 is an exploded perspective view of the bone anchor assembly of FIG. 1, according to some embodiments;

FIG. 3 is a side plan view of a proximal portion of an example of an anchor member forming part of the bone anchor assembly of FIG. 1, according to some embodiments;

FIG. 4 is another side plan view of the proximal portion of the anchor member of FIG. 3, according to some embodiments;

FIG. 5 is a side sectional view of the proximal portion of the anchor member of FIG. 4, according to some embodiments;

FIG. 6 is a side sectional view of an example of a receiver forming part of the bone anchor assembly of FIG. 1, according to some embodiments;

FIG. 7 is a bottom plan view of the receiver of FIG. 6, according to some embodiments;

FIG. 8 is a perspective view of an example of a compression element forming part of the bone anchor assembly of FIG. 1, according to some embodiments;

FIG. 9 is another perspective view of the compression element of FIG. 8, according to some embodiments;

FIG. 10 is a bottom plan view of the compression element of FIG. 8, according to some embodiments;

FIG. 11 is a side sectional view of the bone anchor assembly of FIG. 1, according to some embodiments;

FIG. 12 is a perspective view of another example of a bone anchor assembly, according to some embodiments;

FIG. 13 is an exploded perspective view of the bone anchor assembly of FIG. 12, according to some embodiments;

FIG. 14 is a side sectional view of the bone anchor assembly of FIG. 12, according to some embodiments; and

FIG. 15 is another side sectional view of the bone anchor assembly of FIG. 12, according to some embodiments.

### DETAILED DESCRIPTION OF A PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The modular bone anchor assembly and related methods disclosed herein boasts a variety of inventive features and components that warrant patent protection, both individually and in combination.

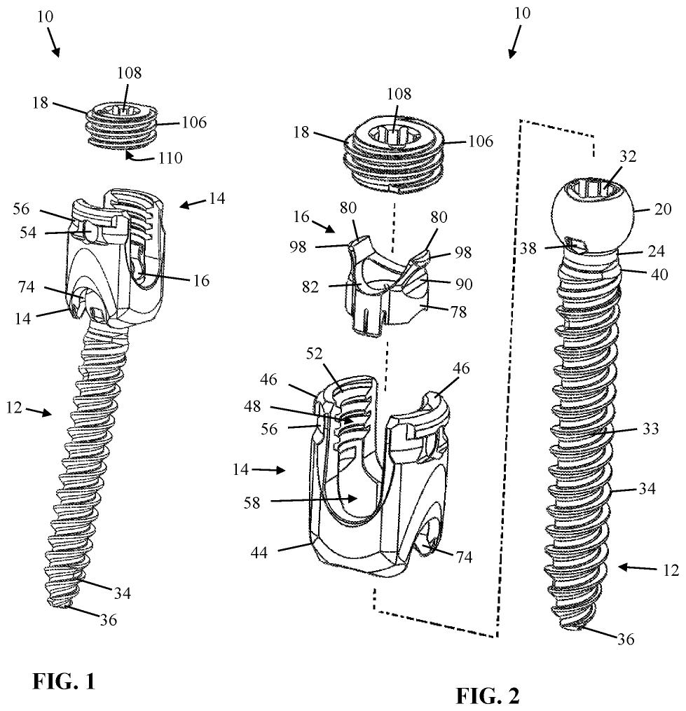

FIGS. 1-2 illustrate an example of a bone anchor assembly 10 according to some embodiments. By way of example only, the bone anchor assembly 10 of the present embodiment may include an anchor member 12, a receiver 14, compression element 16, and a locking element 18. In some embodiments, the bone anchor assembly 10 may be configured to accept an elongate stabilizer (i.e. a rod 5) into the receiver 14, which forms a multi-axial locked relationship with the anchor member 12 (e.g., a screw, hook, nail, staple, etc.). The anchor member 12 may be advantageously capable of being either top loaded or bottom loaded into a receiver 14 and the receiver 14 may be rotated into a position for holding the elongate stabilizer with the compression member 16 that captures a spherical shaped head of the anchor member 12 at one of many possible angles. The elongate stabilizer may be loaded into the receiver 12, unto the compression member 16, and held in that position by a locking element 18 that threads into or onto the receiver 14 and secures the construct in the desired position. In some embodiments, the bone anchor assembly 10 of the present disclosure may be substantially similar to the anchor assemblies described in commonly owned U.S. patent application Ser. No. 16/049,674, filed on Jul. 30, 2018 and entitled “Spinal Stabilization System Including Bottom Loading Wide Angulation Polyaxial Rod Anchor Assemblies,” the complete disclosure of which is hereby incorporated by reference into this disclosure as if set forth fully herein (and is attached as Appendix A to the Specification).

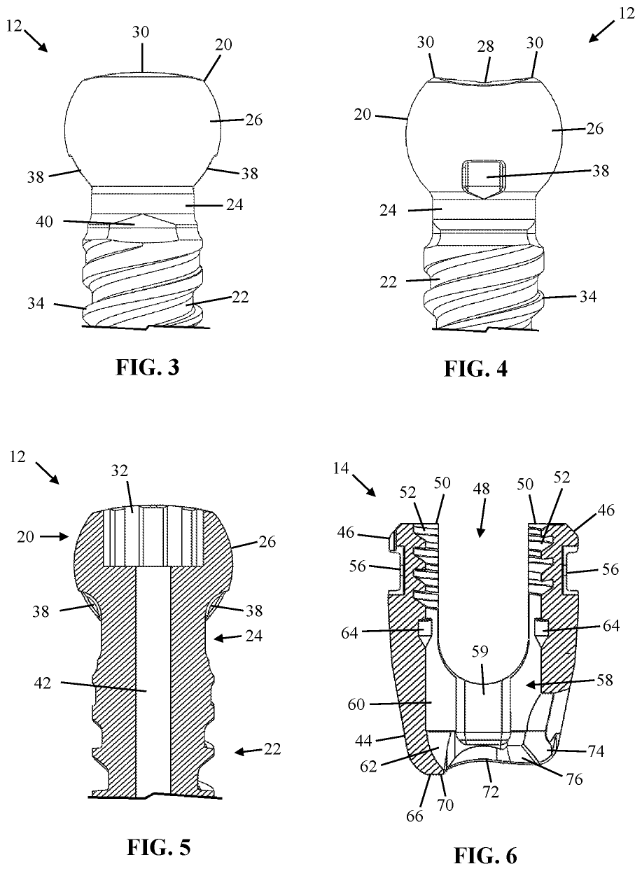

In some embodiments, the anchor member 12 may include a proximally oriented head portion 20, a distally oriented elongated shank 22 extending axially from said head 20 along a longitudinal axis, and a neck portion 24 positioned between the head 20 and the shank 22. By way of example only, FIGS. 3-5 illustrate a proximal portion of the anchor member 12, including the head 20, neck 24, and a proximal portion of the shank 22. In some embodiments, the head 20 may comprise a spherically shaped head 20 having a curved outer surface 26 and a proximal head recess 28 that truncates the spherical shape of the head 20. In some embodiments, the head recess 28 comprises a channel. In some embodiments, the channel is generally perpendicular to the longitudinal axis of the anchor 12. In some embodiments, the head recess 28 may have a concave curvature (see, e.g., FIG. 4) resulting in an outer perimeter having a pair of opposing raised portions 30 on either side of the channel. In some embodiments, the head 20 further comprises a drive feature 32 positioned within the head recess 28. The neck 24 may have a diameter of less than a diameter of the head 20. In some embodiments, the shank 22 includes a bone engagement feature. In some embodiments, the bone engagement feature comprises a helical thread 34 extending at least substantially the length of the shank 22, the helical thread 34 being configured to engage bone. In some embodiments, the distal end 36 of the shank 22 may comprise a narrow tip to facilitate entry into bone. In some embodiments, at least a portion of the shank 22 may comprise a feature to affix to a separate component having a bone engagement feature.

In some embodiments, the head 20 may further comprise one or more distal recesses 38. In some embodiments, the head 20 may comprise at least a pair of opposing distal recesses 38 positioned at a base of the head 20 near an interface with the neck 24. In some embodiments, the distal recesses 38 are cut into the head 20 generally perpendicular to the longitudinal axis. In some embodiments, each distal recess 38 includes a pair of sidewalls 39. As will be explained in further detail below, the distal recesses 38 may function to enable a keyed insertion of the anchor member 12 into the receiver 14.

In some embodiments, the bone anchor 12 may further comprise a relief 40. The bone anchor 12 may comprise two or more reliefs 40. The two or more reliefs 40 may be on opposing sides of the bone anchor 12. The relief 40 may be within the neck 24, the shank 22, or both. The relief 40 may increase the span at which the bone anchor 12 can rotate with respect to the receiver 14. The neck 24 may be configured to fit within the cavity slot 74 of the receiver 14 when the receiver axis and the longitudinal axis of the shank are generally coplanar and wherein the relief 40 is generally parallel to the cavity slot 74.

In some embodiments, the anchor member 12 may be cannulated such that the shank comprises an inner lumen 42 extending between the head 20 and the distal tip 36 of the shank 22.

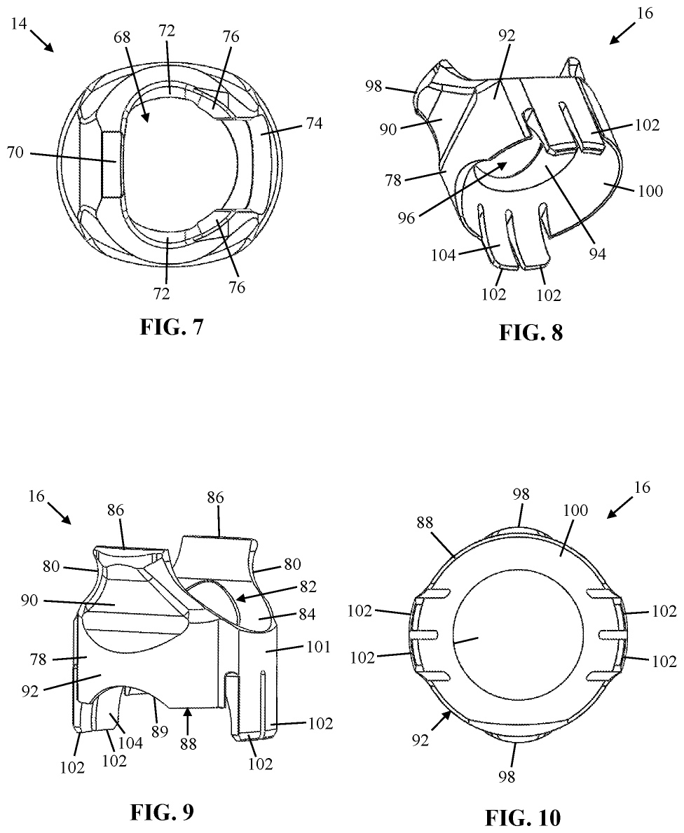

In some embodiments, per FIGS. 1-2 and 6-7, the receiver 14 may have a generally squared-off U-shaped profile with a partially cylindrical inner profile and a substantially faceted cylindrical outer profile. Alternatively, the outer profile could also be of another configuration, for example, curved, faceted, or rectilinear.

In some embodiments, the receiver 14 includes a base 44 and pair of spaced and generally parallel arms 46 that form an open generally U-shaped channel 48 therebetween that may be open at proximal ends 50 of the arms. In some embodiments, the receiver arms 46 each include radially inward or interior surfaces that have a discontinuous guide and advancement structure 52 mateable with threaded surface 106 on the locking element 18. The guide and advancement structure 52 is shown as interrupted internal threads which mate with external threads on the locking element 18, but, more particularly may act as a buttress thread, a square thread, a reverse angle thread, a partial helically wound flange form configured to mate under rotation with a similar structure on the locking element 18 or other thread like or non-thread like helically wound advancement structure for operably guiding under rotation and advancing the locking element 18 downward between the receiver arms 46 and having such a nature as to resist (or at least not to contribute to) splaying of the receiver arms 46 when the locking element 18 is advanced there between. In some embodiments, the base 44 and the arms 46 forming the U-channel 48 may be comprised of one or more components. For example, while in the illustrated embodiments, the base 44 and the arms 46 comprise a single component, in other embodiments, the base 44 may be a separate component in rotational or translational articulation with the arms 46 and the U-shaped channel 48. In some embodiments, the receiver arms 46 include opposed tool engaging divots 54 formed on or through outer surfaces of such arms as well as opposed tool engaging grooves 56. The divots 54 and/or grooves 56 may be used for holding the receiver 14 during assembly with the anchor member 12, during the implantation of the shank 22 of the anchor member 12 into a vertebra or other bone and assembly with the rod 5 and the locking element 18. In some embodiments, the tool-receiving divots 54 or grooves 56 may be configured in a variety of shapes and sizes and be disposed at other locations on the receiver arms 46.

In some embodiments, the receiver 14 further includes a chamber or cavity 58 located within the base 44 communicating with and located beneath or distal to the U-shaped channel 48. In some embodiments, the chamber or cavity 58 may be defined in part by an inner substantially cylindrical surface 60 and further includes a substantially spherical seating surface portion 62 configured to seat or receive the head 20 of the anchor member 12. In some embodiments, the cylindrical surface 60 that defines a portion of the cavity 58 opens upwardly into the U-shaped channel 48 and includes opposing retention features 64 (shown by way of example only in FIG. 6) configured to receive at least a portion of the protrusions 98 on the upstanding arms 80 of the compression element 16 therein to retain the compression element 16 within the receiver 14. In some embodiments, the retention features 64 may include partial radial grooves on each side of the channel 48. In some embodiments, the retention features 64 may comprise grooves, holes, fins, bosses, projections or other structures that may be utilized for the purpose of preventing disassembly of the compression element 16 from the receiver 14. In some embodiments, the cavity 58 further includes one or more longitudinal recesses 59 configured to receive the one or more radial protrusions 101 of the compression element 16 to prevent rotation of the compression element 16 relative to the receiver 14 during and after assembly of the bone anchor assembly 10.

The substantially spherical seating surface portion 62 is located below or distal to the inner cylindrical surface 60 and is sized and shaped for mating with the curved outer surface 26 of the head 20 of the anchor member 12. The base 44 of the receiver 14 further comprises a distal surface 66 and a distal or terminal opening 68 in communication with the seating surface portion 62 and the cavity 58. The distal opening 68 is substantially coaxially aligned with respect to a rotational axis of the receiver 14 (e.g., an axis extending longitudinally through the U-shaped channel 46 and cavity 58 in a proximal to distal direction). The distal opening 68 has a generally circular shape that may be partially occluded by a keyed feature or shoulder 70 extending into the circular shape of the distal opening 68. By way of example, the shoulder 70 may be configured to aid in retention of the anchor member 12. In some embodiments, the bottom edge of the terminal opening 68 may include chamfers 72 that allow increased angulation of the shank 22 in preferred directions when assembled into receiver 14. By way of example, the chamfers 72 do not encroach on the shoulder 70.

In some embodiments, base 44 of the receiver 14 further includes a cavity slot 74 that communicates with the contoured volume of the cavity 58, as well as the distal opening 68 and a receiver side exterior. In some embodiments, the cavity slot 74 is located opposite the shoulder 70. In some embodiments, the cavity slot 74 is shaped to allow the neck 22 of the anchor member 12 to reside therein. In some embodiments, the receiver 14 further comprises one or more interference elements in the form of protrusions 76 positioned on the bottom edge of the terminal opening 68 at the interface with the cavity slot 74.

In some embodiments, the cavity slot 74 allows the anchor member 12 to assume an entry position during bottom-loading assembly (and/or disassembly) of the anchor assembly 10 where the longitudinal axis of the anchor member 12 is generally perpendicular to the receiver rotational axis, the head recess 28 of the anchor member 12 is aligned and engaged with the shoulder 70 of the receiver 14, and one of the distal recesses 38 of the head 20 is engaged with one of the protrusions 76 of the receiver 14. With the engagement of the protrusion 76 within the distal recess 38 and one of the sidewalls 39 acting as a buttress, the anchor member 12 may be rotated about its longitudinal axis, enabling the neck 24 to rotate or “roll” into the cavity slot 74 and the head 20 of the anchor member 12 to pass both the shoulder 70 and the protrusions 76 into (and/or out of) the contoured volume of the cavity 58. Once the head 20 is seated within the contoured volume of the cavity, the anchor member 12 may be pivoted in many different directions or angles without dissembling from the receiver 14.

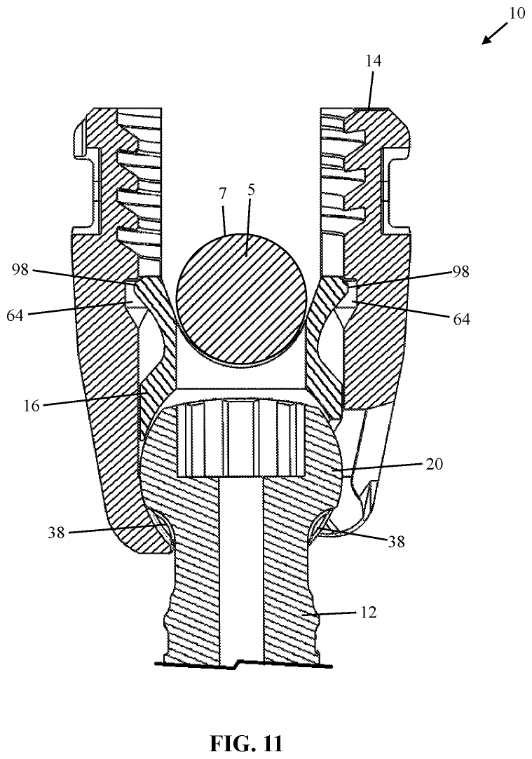

FIGS. 8-10 illustrate an example of a compression element or load ring 16 forming part of the anchor assembly 10 according to some embodiments. By way of example only, the compression element 16 includes a body 78 having a generally circular cross section and a pair of upstanding arms 80 extending in a proximal direction from the body 78. In some embodiments, the body 78 and arms 80 form a generally U-shaped, open channel 82 having a concave or substantially U-shaped proximal-facing bottom rod seating surface 84. By way of example only, the rod seating surface 84 may have a radius substantially conforming or slightly undersized to a radius of the rod 5 (see FIG. 11) and thus is configured to snugly engage the rod 5 in operation. In some embodiments, the arms 80 disposed on either side of the channel 82 may each include a top surface 86 that is parallel to a bottom surface 88. In some embodiments, the bottom surface 88 includes a cutout portion 89 sized and configured to create clearance to allow passage of the neck 24 of the anchor member 12 (for example) during assembly. In some embodiments, each upstanding arm 80 may further include a relief cut 90 designed to thin the arms 80 and allow them to flex slightly during assembly of compression element 16 into the receiver 14. By way of example, the compression element 16 may include a generally cylindrical outer surface 92 and an inner cylindrical wall 94 defining a central through-bore 96 extending along a central axis C of the compression element 16. In some embodiments, the outer surface 92 may further include outward-facing protrusions 98 on each of the upstanding arms 80 proximal to the top surfaces 86. The top surface 86 and the bottom surface 88 are disposed perpendicular to the axis C. In some embodiments, the compression element 16 further comprises a distal facing curved or spherical inner surface 100 extending between the inner cylindrical wall 94 and the bottom surface 88. By way of example, the curved or spherical inner surface 100 may be sized and shaped to frictionally engage and mate with the outer spherical surface 26 of the head 20 of the anchor member 12.

By way of example only, the cylindrical outer surface 92 of the compression element 16 may have a diameter slightly smaller than a diameter between crests of the threads of the receiver guide and advancement structure 52, but the protrusions 98 form a diameter that is slightly larger than the diameter between crests of the threads of the guide and advancement structure 52 of the receiver 14. This allows the compression element 16 to be top-loaded into the receiver 14 by means of the upstanding arms 80 deflecting slightly inward during assembly as the compression element 16 is advanced into the receiver 14 until the protrusions 98 reach the retention features (i.e. recesses) 64 of the receiver 14 and are allowed to spring outward and engage the retention features 64, for example as shown in FIG. 11. At this stage, the compression element 16 is detained inside the receiver 14 and prevented from coming back out through the top of the receiver 14, while also being allowed some ability to translate distally under load in order to press upon the head 20 of the anchor member 12, with or without deflection of the arms 80.

In some embodiments, the cylindrical surface 92 of the compression element 16 has a diameter and a height measured from the top surface 86 to the bottom surface 88 and is sized such that the compression element 16 may be received within the receiver 14 adjacent the inner cylindrical surface 60 and below the guide and advancement structure 52, but the bottom surface 88 thereof does not engage the spherical seating surface 62 of the receiver 14. When fully assembled, with the anchor element 12 coupled with the receiver 14, a rod or connecting member 5 is inserted through the U-shaped channel 48 of the receiver 14 until it is pressing on the rod seating surface 84 of the compression element 16. This distal force exerted on and by the rod 5 causes the spherical inner surface 100 of the compression element 16 to frictionally engage the curved outer surface 26 of the head 20 of the anchor element 12 which in turn presses upon the seating surface portion 62 of the receiver 14. It is foreseen that the compression element 16 may take on a variety of different configurations and means of retention inside the receiver 14.

In some embodiments, the compression element 16 may include additional features configured to increase the frictional interface between the head 20 of the anchor member 12 and the receiver 14. By way of example, in some embodiments the compression element 16 may include at least one radial protrusion 101 extending radially from the cylindrical surface 92 configured engage with a longitudinal recess 59 of the receiver 14 to prevent rotation of the compression element 16 relative to the receiver 14 during and after assembly of the bone anchor assembly 10. Additionally, compression element 16 may further include flexible flanges 102 extending distally from the bottom surface 88, which may also extend from or constitute a distal extension of the radial protrusion 101. In some embodiments, the compression element 16 may include a plurality of flexible flanges 102 extending distally from the bottom surface 88. The flexible flanges 102 comprise an inner mating surface 104 configured to engage the curved outer surface 26 of the head 20 of the anchor member 12. In some embodiments, the flexible flanges 102 do not extend past an equator of the head 20. In some embodiments, the flexible flanges 102 are sized and configured to extend past the equator of the head 20 to maximize the surface area interface between the compression element 16 and the head 20, and creating a friction fit interaction before final tightening of the construct. This can be advantageous to control the positioning of the receiver 14 and/or bone anchor during assembly of a rod-based fusion construct. It should be understood that the compression member 16 may be shaped in ways other than the illustrated embodiments so long as it has a spherical receiving surface for contacting the head of the anchor member, is able to be retained (e.g., or “nest”) inside the receiver, and is capable of at least some downward translation in order to exert force onto the screw head to lock the screw angle relative to the receiver.

With further reference to FIGS. 1-2, the locking element 18 may further include at least a threaded surface 106 and a compression drive feature 108. In some embodiments, the compression drive feature 108 extends completely through the locking element 18, but in other embodiments, the compression drive feature 108 may comprise a blind recess in upper surface of the locking element 18. The locking element 18 may engage a longitudinal connecting member 5, such as a rod having a cylindrical surface 7 shown in FIG. 11. In some embodiments, the locking element 18 includes a compression bottom surface 110 that presses against the connecting member 5 that in turn presses upon the compression element 16 that in turn presses the head 20 of the anchor member 12 into fixed frictional contact with the receiver 14, so as to lock or fix the connecting member 5 relative to the vertebra (not shown). In some embodiments, the receiver 14 and the anchor member 12 cooperate in such a manner that the receiver 14 and the anchor member 12 can be secured at any of a plurality of angles, articulations, or rotational alignments relative to one another and within a selected range of angles both from side to side and from front to rear, to enable flexible or articulated engagement of the receiver 14 with respect to the anchor member 14 until both are locked or fixed relative to each other near the end of an implantation procedure.

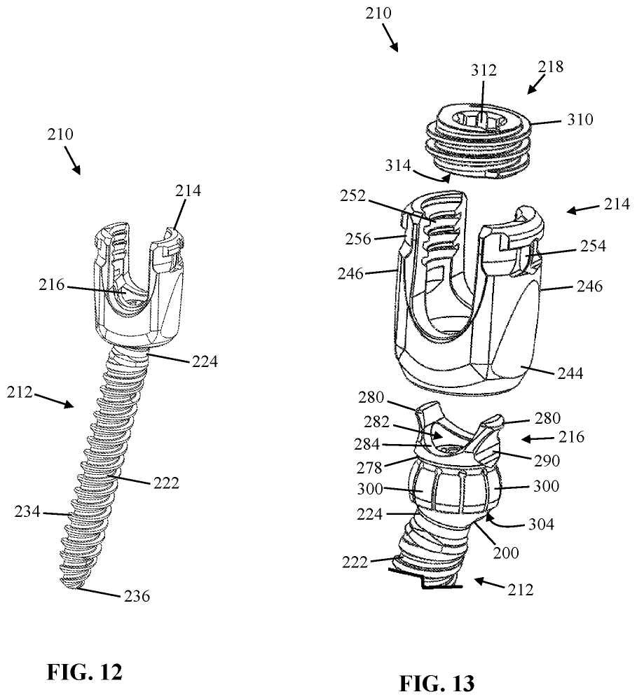

FIGS. 12-15 illustrate an example of a bone anchor assembly 210 according to some embodiments. In some embodiments, the bone anchor assembly 210 may include an anchor member 212, a receiver 214, compression element 216, and a locking element 218. In some embodiments, the bone anchor assembly 210 may be configured to accept an elongate stabilizer (i.e. a rod 5) into the receiver 214, which forms a multi-axial locked relationship with the anchor member 212 (e.g., a screw, hook, nail, staple, etc.). The anchor member 212 may be advantageously capable of being either top loaded or bottom loaded into a receiver 214 and the receiver 214 may be rotated into a position for holding the elongate stabilizer 5 with the compression member 216 that captures a spherical shaped head of the anchor member 212 at one of many possible angles. The elongate stabilizer may be loaded into the receiver 212, unto the compression member 216, and held in that position by a locking element 218 that threads into or onto the receiver 214 and secures the construct in the desired position.

In some embodiments, the anchor member 212 may be anchor member 12 described above. In some embodiments, the anchor member 212 may include a proximally oriented head portion 220, a distally oriented elongated shank 222 extending axially from said head 220 along a longitudinal axis, and a neck portion 224 positioned between the head 220 and the shank 222. In some embodiments, the head 220 may comprise a spherically shaped head 220 having a curved outer surface. In some embodiments, the head 220 further comprises a drive feature 232 positioned at the proximal end of the head. The neck 224 may have a diameter of less than a diameter of the head 220. In some embodiments, the shank 222 includes a bone engagement feature. In some embodiments, the bone engagement feature comprises a helical thread 234 extending at least substantially the length of the shank 222, the helical thread 234 being configured to engage bone. In some embodiments, the distal end 236 of the shank 222 may comprise a narrow tip to facilitate entry into bone. In some embodiments, at least a portion of the shank 222 may comprise a feature to affix to a separate component having a bone engagement feature. In some embodiments, the anchor member 212 may be cannulated such that the shank comprises an inner lumen 242 extending between the head 220 and the distal tip 236 of the shank 222.

In some embodiments, the receiver 214 may have a generally squared-off U-shaped profile with a partially cylindrical inner profile and a substantially faceted cylindrical outer profile. Alternatively, the outer profile could also be of another configuration, for example, curved, faceted, or rectilinear.

In some embodiments, the receiver 214 includes a base 244 and pair of spaced and generally parallel arms 246 that form an open generally U-shaped channel 248 therebetween that may be open at proximal ends of the arms. In some embodiments, the receiver arms 246 each include radially inward or interior surfaces that have a discontinuous guide and advancement structure 252 mateable with threaded surface 310 on the locking element 218. The guide and advancement structure 252 is shown as interrupted internal threads which mate with external threads on the locking element 218, but, more particularly may act as a buttress thread, a square thread, a reverse angle thread, a partial helically wound flange form configured to mate under rotation with a similar structure on the locking element 218 or other thread like or non-thread like helically wound advancement structure for operably guiding under rotation and advancing the locking element 218 downward between the receiver arms 246 and having such a nature as to resist (or at least not to contribute to) splaying of the receiver arms 246 when the locking element 218 is advanced there between. In some embodiments, the base 244 and the arms 246 forming the U-channel 248 may be comprised of one or more components. For example, while in the illustrated embodiments, the base 244 and the arms 246 comprise a single component, in other embodiments, the base 244 may be a separate component in rotational or translational articulation with the arms 246 and the U-shaped channel 248. In some embodiments, the receiver arms 246 include opposed tool engaging divots 254 formed on or through outer surfaces of such arms as well as opposed tool engaging grooves 256. The divots 254 and/or grooves 256 may be used for holding the receiver 214 during assembly with the anchor member 212, during the implantation of the shank 222 of the anchor member 212 into a vertebra or other bone and assembly with the rod 5 and the locking element 218. In some embodiments, the tool-receiving divots 254 or grooves 256 may be configured in a variety of shapes and sizes and be disposed at other locations on the receiver arms 246.

In some embodiments, the receiver 214 further includes a chamber or cavity 258 located within the base 244 communicating with and located beneath or distal to the U-shaped channel 248. In some embodiments, the chamber or cavity 258 may be defined in part by an inner substantially cylindrical surface 260 and further includes a substantially spherical seating surface portion 262 configured to seat or receive the curved outer surface 308 of the flexible flanges 300 of the compression element 216. In some embodiments, the cylindrical surface 260 that defines a portion of the cavity 258 opens upwardly into the U-shaped channel 248 and includes opposing retention features 264 configured to receive at least a portion of the protrusions 298 on the upstanding arms 280 of the compression element 216 therein to retain the compression element 216 within the receiver 214. In some embodiments, the retention features 264 may include partial radial grooves on each side of the channel 248. In some embodiments, the retention features 264 may comprise grooves, holes, fins, bosses, projections or other structures that may be utilized for the purpose of preventing disassembly of the compression element 216 from the receiver 214.

The substantially spherical seating surface portion 262 is located below or distal to the inner cylindrical surface 260 and is sized and shaped for mating with the curved outer surface 308 of the flexible flanges 300 of the compression element 216. The base 244 of the receiver 14 further comprises a distal surface 266 and a distal or terminal opening 268 in communication with the seating surface portion 262 and the cavity 258. The distal opening 268 is substantially coaxially aligned with respect to a rotational axis of the receiver 214 (e.g., an axis extending longitudinally through the U-shaped channel 246 and cavity 258 in a proximal to distal direction). By way of example only, the distal opening 268 may have a generally circular shape and a radius that is large enough to enable passage of the head 220 of the anchor member 212 therethrough to enable for bottom-loading assembly of the anchor assembly 210. In some embodiments, the bottom edge of the terminal opening 268 may include chamfers 272 that allow increased angulation of the shank 222 in preferred directions when assembled into receiver 214.

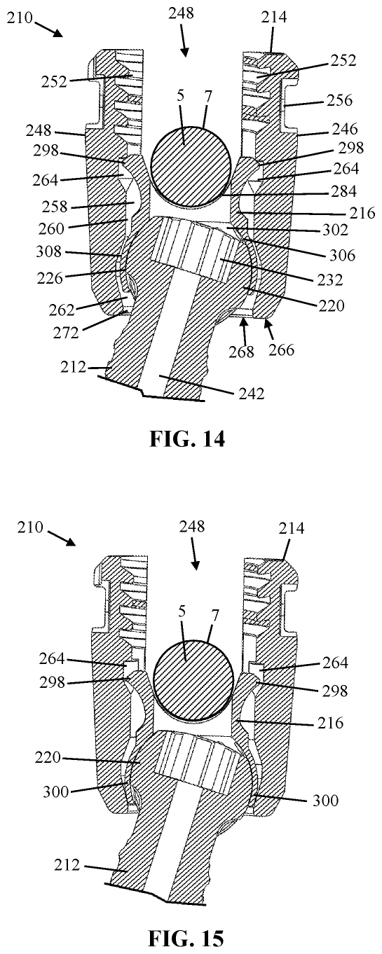

By way of example only, the compression element 216 includes a body 278 having a generally circular cross section, a pair of upstanding arms 280 extending in a proximal direction from the body 278, and a distal engagement portion 281 extending distally from the body 278. In some embodiments, the body 278 and arms 280 form a generally U-shaped, open channel 282 having a substantially U-shaped bottom rod seating surface 284. By way of example only, the rod seating surface 284 may have a radius substantially conforming or slightly undersized to a radius of the rod 5 (see FIGS. 14-15) and thus is configured to snugly engage the rod 5 in operation. In some embodiments, the arms 280 disposed on either side of the channel 282 may each include a top surface that is parallel to a bottom surface. In some embodiments, each upstanding arm 280 may further include a relief cut 290 designed to thin the arms 280 and allow them to flex slightly during assembly of compression element 216 into the receiver 214. By way of example, and similar to the compression element 16 described above, the compression element 216 may include a generally cylindrical outer surface and an inner cylindrical wall defining a central through-bore extending along a central axis C of the compression element 216.

In some embodiments, the outer surface may further include protrusions 298 on each of the upstanding arms 280 proximal to the top surfaces. The top surface and the bottom surface are disposed perpendicular to the axis C.

By way of example only, the distal engagement portion 281 is configured to receive at least a portion of the head 220 of the anchor member 212 therein. In some embodiments, the distal engagement portion 281 is configured to receive a substantial portion of the head 220 of the anchor member 212 therein. In some embodiments, the distal engagement portion 281 comprises a plurality of curved, flexible flanges 300 arranged in an annular ring configuration and defining a cavity 302 therein sized and configured for receiving the head 220 of the anchor member 212 therein. In some embodiments, the distal ends of the flexible flanges 300 together form an annular opening 304 into the cavity 302. By way of example only, the flanges 300 are each curved such that each flange has an inner surface 306 having a concave curvature and an outer surface 308 having a convex curvature, and further such that the maximum diameter of the cavity 302 is greater than the diameter of the opening 304. By way of example, the diameter of the opening 304 in a first, nonexpanded state is smaller than the maximum diameter of the head 220 such that, absent expansion of the opening 304, the head 220 is prevented from entering or exiting the cavity 302. By way of example, the curved or spherical inner surface 306 may be sized and shaped to engage the outer spherical surface 226 of the head 220 of the anchor member 212.

In some embodiments, the annular ring of flexible flanges 300 is configured to flex or expand radially outward to widen the opening 304 and allow passage of the head 220 of the anchor member 212 through the expanded opening 304 in response to a proximal or upward force exerted by the head 220 on the compression element 216, for example when the anchor member 212 is being coupled to the receiver 214 through a bottom-loading coupling process. To facilitate this, the compression element 216 translates proximally or upward within the receiver 214, and the protrusions 298 translate proximally or upward within the retention features 264 described above. As illustrated by way of example only in FIG. 14, this proximal translation shifts the compression element 216 a sufficient distance within the receiver cavity 258 to provide enough clearance for the flexible flanges 300 to radially expand within the cavity 258 to enable passage of the head 220 through the opening 304 as the head 220 is forced through.

In some embodiments, once the head 220 of the anchor member 212 has been inserted into the receiver 214 (by virtue of coupling with the compression element 216), a distal oriented or downward force applied to the anchor member 212 causes the compression element 216 to translate distally within the receiver 214 (and correspondingly causes the protrusions 298 to translate distally within the retention features 264). As a result of this distal translation, the flexible flanges 300 are blocked from flexing or expanding radially outward by virtue of the inner cylindrical surface 260 of the receiver cavity 258, as shown by way of example only in FIG. 15. Thus, the head 220 of the anchor member 212 is captured within the cavity 302 of the compression element 216 and the anchor member 212 is securely coupled with the receiver 214.

By way of example only, the cylindrical outer surface of the compression element 216 may have a diameter slightly smaller than a diameter between crests of the threads of the receiver guide and advancement structure 252, but the protrusions 298 form a diameter that is slightly larger than the diameter between crests of the threads of the guide and advancement structure 252 of the receiver 214. This allows the compression element 216 to be top-loaded into the receiver 214 by means of the upstanding arms 280 deflecting slightly inward during assembly as the compression element 216 is advanced into the receiver 214 until the protrusions 298 reach the retention features (i.e. recesses) 264 of the receiver 214 and are allowed to spring outward and engage the retention features 264, for example as shown in FIG. 14. At this stage, the compression element 216 is detained inside the receiver 214 and prevented from coming back out through the top of the receiver 214, while also being allowed some ability to translate distally under load in order to press upon the head 220 of the anchor member 212, with or without deflection of the arms 280.

In some embodiments, the cylindrical surface of the compression element 216 has a diameter and a height measured from the top surface to the bottom surface and is sized such that the compression element 216 may be received within the receiver 214 adjacent the inner cylindrical surface 260 and below the guide and advancement structure 252, but the bottom surface thereof does not engage the spherical seating surface 262 of the receiver 214. When fully assembled, with the anchor element 212 coupled with the receiver 214, a rod or connecting member 5 is inserted through the U-shaped channel 248 of the receiver 214 until it is pressing on the rod seating surface 284 of the compression element 216. This distal force exerted on and by the rod 5 causes the spherical inner surface 306 of the compression element 216 to engage the curved outer surface 226 of the head 220 of the anchor element 212 which in turn presses upon the seating surface portion 262 of the receiver 214.

In some embodiments, the locking element 218 may further include at least a threaded surface 310 and a compression drive feature 312. In some embodiments, the compression drive feature 312 extends completely through the locking element 218, but in other embodiments, the compression drive feature 312 may comprise a blind recess in upper surface of the locking element 218. The locking element 218 may engage a longitudinal connecting member 5, such as a rod having a cylindrical surface 7 shown in FIGS. 14-15. In some embodiments, the locking element 218 includes a compression bottom surface 314 that presses against the connecting member 5 that in turn presses upon the compression element 216 that in turn presses the head 220 of the anchor member 212 into fixed frictional contact with the receiver 214, so as to lock or fix the connecting member 5 relative to the vertebra (not shown). In some embodiments, the receiver 214 and the anchor member 212 cooperate in such a manner that the receiver 214 and the anchor member 212 can be secured at any of a plurality of angles, articulations, or rotational alignments relative to one another and within a selected range of angles both from side to side and from front to rear, to enable flexible or articulated engagement of the receiver 214 with respect to the anchor member 214 until both are locked or fixed relative to each other near the end of an implantation procedure.

Any of the features or attributes of the above the above described embodiments and variations can be used in combination with any of the other features and attributes of the above described embodiments and variations as desired.

From the foregoing disclosure and detailed description of certain preferred embodiments, it is also apparent that various modifications, additions and other alternative embodiments are possible without departing from the true scope and spirit. The embodiments discussed were chosen and described to provide the best illustration of the principles of the present invention and its practical application to thereby enable one of ordinary skill in the art to utilize the invention in various embodiments and with various modifications as are suited to the particular use contemplated. All such modifications and variations are within the scope of the present invention as determined by any and all claims deriving from this disclosure when interpreted in accordance with the benefit to which they are fairly, legally, and equitably entitled.

What is claimed is:

1. A load ring configured for use with a bone anchor assembly having a rod receiver and an anchor member, the anchor member having a spherical head, a bone-engaging shank, and a neck portion between the head and shank, the load ring comprising:
a body sized and configured to nest within the rod receiver and having a at least one deflectable upstanding arm extending in a proximal direction from the body, the body further including a U-shaped, open channel having a concave proximal-facing rod seating surface configured to seat a cylindrical stabilizing rod therein, the body further comprising a distal-facing spherical inner surface configured to frictionally engage the spherical head of the anchor member; and
at least one outward-facing protrusion on the upstanding arm configured to engage a protrusion-receiving recess on an inner surface of the rod receiver upon assembly of the load ring with the rod receiver;
wherein the at least one upstanding arm is configured to deflect inward from an initial state to a deflected state to enable advancement of the load ring into the rod receiver in a proximal to distal direction; and
wherein the at least one upstanding arm is configured to return from the deflected state to the initial state when the load ring has advanced into the rod receiver such that the outward-facing protrusion engages the protrusion-receiving recess, thereby securing the load ring within the rod receiver.

2. The load ring of claim 1, further comprising at least one radial protrusion extending radially from the body and configured to engage the receiver to prevent rotation of the load ring relative to the rod receiver during or upon assembly with the rod receiver.

3. The load ring of claim 1, further comprising at least one flexible flange extending distally from the body and having an inward facing spherical surface.

4. The load ring of claim 3, wherein the at least one flexible flange is configured to extend past an equator of the spherical head of the anchor member upon assembly of the bone anchor assembly.

5. The load ring of claim 3, wherein the at least one flexible flange is configured such that the flexible flange does not extend past an equator of the spherical head of the anchor member upon assembly of the bone anchor assembly.

6. The load ring of claim 1, wherein the body further comprises a bottom surface, and wherein the bottom surface includes a cutout portion sized and configured to allow passage of the neck of the anchor member during assembly of the bone anchor assembly.
